(19) United States
(12) Patent Application Publication
(10) Pub. No.: US 2011/0082496 A1
(43) Pub. Date: Apr. 7, 2011

(54) Endovascular device and clotting system for the repair of vascular defects and malformations

(76) Inventor: Ashish Awasthi, Cranbury, NJ (US)

(21) Appl. No.: 12/587,183

(22) Filed: Oct. 2, 2009

(51) Int. Cl. A61B 17/08

(52) U.S. Cl. 606/213

(57) ABSTRACT

A device for sealing damaged and defective vessels utilizes a surgical wire or similar appliance positioned and housed within a protective sheath, such as a catheter or needle. Stable thrombin and non-thrombin based solutions, gels, or biological glues are then used to coat the tip of a surgical appliance, such as surgical wires and trocars, with concentrations sufficient to cause clotting. The protective sheath around the wire and a one way valve provides a barrier which prevents clotting material to insinuate itself into healthy tissue. The protective catheter containing the wire is then inserted into the body of the patient and directed to the damaged site. The wire is then partially extended out of the sheath to allow the clotting material to be applied and deposited onto the damaged vessel, thereafter the wire and sheath are safely withdrawn from the body of the patient.

### BACKGROUND OF THE INVENTION

The device and system of the present invention addresses the sealing of damaged or defective tissue, whether congenital or acquired, e.g. vascular punctures, aneurysms, pseudoaneurysms, fistulas, ateriovenous malformations (AVM), and similar lumens. It is critical that such tissue be treated effectively and completely reduce and then stop blood flow from such tissues and initiate the body's own hemostatic processes. Co-pending application Ser. No. 12/462,391 discloses the method in which the system is used.

Thrombin is a clotting factor which reacts with soluble fibrinogen, a blood plasma protein, converting it to fibrin. Fibrin is the basis and is essential for clotting of blood. As a result, thrombin has found widespread usage in surgical closure and hemostatic methods. In addition, thrombin is used in many surgical glues for obtaining hemostatsis on surgical incisions or puncture locations. Such surgical glues are directly applied to the damaged or defective tissue or by application on the skin to stop blood flow following surgery.

Additionally, thrombin solutions have been used for treating post-procedure complications, including pseudoaneurysms, by direct injection into the false lumen of these defects to stop blood flow and coagulate the defect, with spontaneous resolution over time.

Other non-thrombin based procedures have been used to shrink atriovenous defects in high risk areas such as the brain, before treatment with other options. For example, these techniques employ a catheter with a balloon at one end to stop spillage of the material, as it is highly thrombogenic. Small aneurysms in difficult to reach areas of the brain have been treated by coils in order to cause the aneurysms to clot.

Successful as such techniques may be, they carry a significant risk of initiating clotting in normal tissue as a result of spillage of thrombin and other non-thrombin solutions. Additionally, thrombin injections are not used for intravascular procedures, due to the extreme and dangerous risk of clotting. Coils are, on occasion, difficult to deliver and thus unable to provide the required treatment.

### SUMMARY OF THE INVENTION

Thus, the device and system of the present invention is directed towards addressing the disadvantages and limitations of prior clotting material delivery techniques and thus improve treatment of damaged and defective vessels which must be sealed.

It is thus the object of the present invention to provide a device which allows for the effective and ready delivery of clotting material to defects within blood vessels and other tissues in a safe and efficient manner.

It is a further object of the present invention to provide a device for sealing damaged and defective vessels which provides for a barrier between blood/tissue and the clotting material until the material reaches the targeted defective site.

It is still another object of the present invention to provide a device for sealing damaged and defective vessels which allows for delivery of sufficient clotting material to be provided to the defective site.

It is another object of the present invention to provide a device for sealing damaged vessels such that the clotting material does not detach and embolize, thereby straying to an undesired location which would cause dangerous clotting and other serious problems.

These and other objects are accomplished by the present invention, a device for sealing damaged and defective vessels which utilizes an interventional wire or similar appliance positioned and housed within a protective sheath, such as a catheter or needle. The protective catheter has a valve at its end preventing the clotting material from coming in contact with blood until required. Stable thrombin and non-thrombin based solutions, gels, or biological glues are then used to coat the tip of a surgical appliance, such as an interventional wire or trocar, with concentrations sufficient to cause clotting. The protective sheath with a one way valve around the wire provides a barrier which prevents clotting material to insinuate itself into healthy tissue. The protective catheter containing the wire is then inserted into the body of the patient and directed to the damaged site. The wire is then partially extended out of the sheath to allow the clotting material to be applied and deposited at the site of damaged vessel, thereafter the wire and sheath are safely withdrawn from the body of the patient.

The novel features which are considered as characteristic of the invention are set forth in particular in the appended claims. The invention, itself, however, both as to its design, construction and use, together with additional features and advantages thereof, are best understood upon review of the following detailed description with reference to the accompanying drawings.

### BRIEF DESCRIPTION OF THE DRAWINGS

FIG. 1 shows the device of the present invention, with its surgical wire positioned within its protective sheath, i.e. a catheter.

FIG. 2 is a cross-sectional view of FIG. 1 and shows the device of the present invention, with one way valve, wire with tip coated with thrombotic material, and a backstop to prevent the wire from coming out of the device.

FIG. 3 is a view similar to FIG. 1, but showing the device of the present invention in operation, as its wire begins travel through and out of the catheter.

FIG. 4 shows an alternate means of controlling the opening and closing of the catheter of the present invention.

FIG. 5 depicts a preliminary step of the method employing the device of the present invention.

FIG. 6 shows a subsequent step of the method employing the device of the present invention.

FIG. 7 shows a later step of the method employing the device of the present invention.

### DETAILED DESCRIPTION OF THE INVENTION

The unique device 1 of the present invention comprises a surgically protective sheath, such as hollow, long, micro-catheter 4, with one way valve 6, as a barrier to prevent the clotting material to be applied and deposited on the damaged vessel/tissue, to come into contact with healthy, normal tissue, blood, and other susceptible areas unrelated to the treatment. An elongated appliance such as a trocar or surgical wire 2 is configured to be positioned within and travel through protective sheath 4 to the damaged tissue to be clotted and closed.

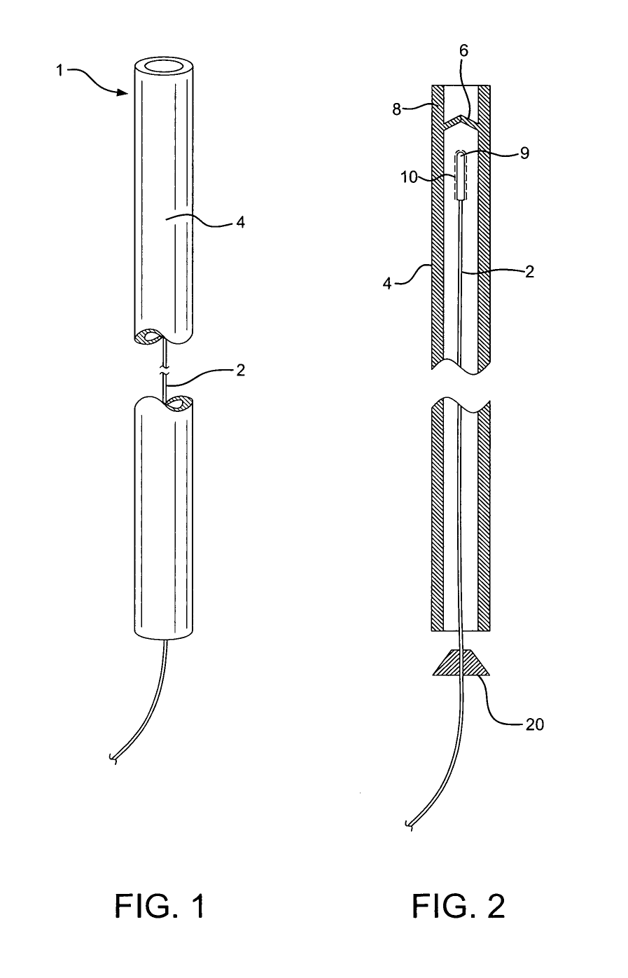

Flexibly, maneuverable wire 2 in the range of 0.014 to 0.026 inches in diameter, is positioned to be advanced through catheter 4, ranging in diameter for 0.018 to 0.035 inches in diameter. (See FIG. 1). Clotting material 10, such as thrombin, biological glues or other sealant, is deposited on tip 9 of wire 2. It is contemplated that approximately 5-10 mm of the end of wire 2 will be coated with clotting material 10, as appropriate for the specific application. Tip 9 can be spiral in configuration, to accommodate more clotting material, as may be appropriate.

Catheter 4 has one way valve 6 at its distal end 8 which is normally closed, but which will permit wire 2, its tip 9 coated with clotting material 10, to pass through and out of the catheter. Thus the clotting material only comes in contact with blood when desired at the treatment site. Further, the clotting material will not come out accidentally prior to reaching the treatment site due to removeable backstop 20, which prevents the tip from moving. (See FIG. 2). Alternatively, catheter 4a can be opened and closed by a petal closure device. That is, it can have two wire strands 12 and 14 woven into its sides. Strands 12 and 14 are connected to biased, rotatable plates 16 and 18 which are normally closed, but which are configured to be opened when the strands are pulled. The opening and closing of distal end 8 of catheter 4 can also be accomplished by other recognized means. The herein invention is not to be considered restricted to the type of catheter closure device which is used.

After tip 9 of wire 2 is coated with clotting material 10, the wire is positioned in catheter 4. A radio opaque marker can be placed near the end of catheter 4, so that its position can be observed when in the body of a patient. A balloon may also be attached near the end of catheter 4 in order to stabilize its placement within the body. It is critical that wire 2 with its clotting material remain closeted within catheter 4 by means of one way valve 6 to avoid clotting material contact with healthy blood and tissue. As previously described, removeable backstop 20 is provided around wire 2 to prevent wire 2 from exiting catheter 4 until such is needed.

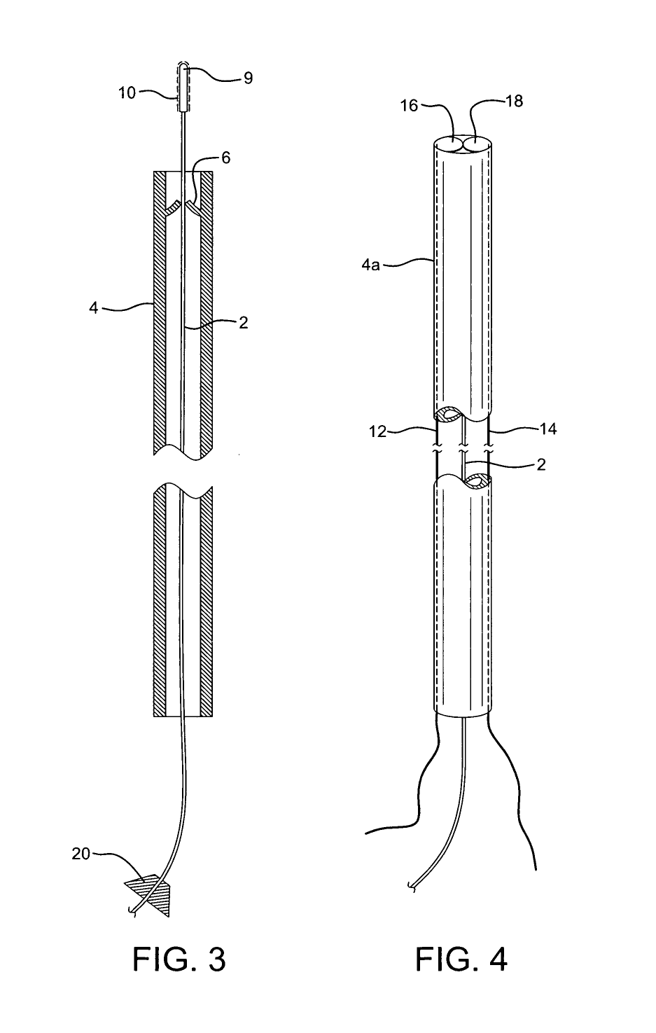

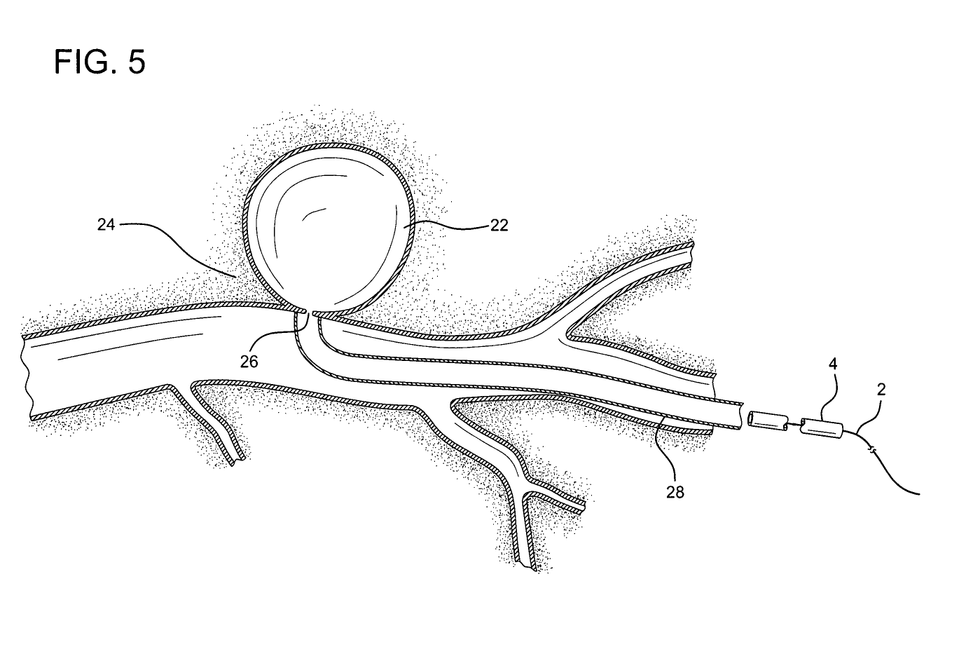

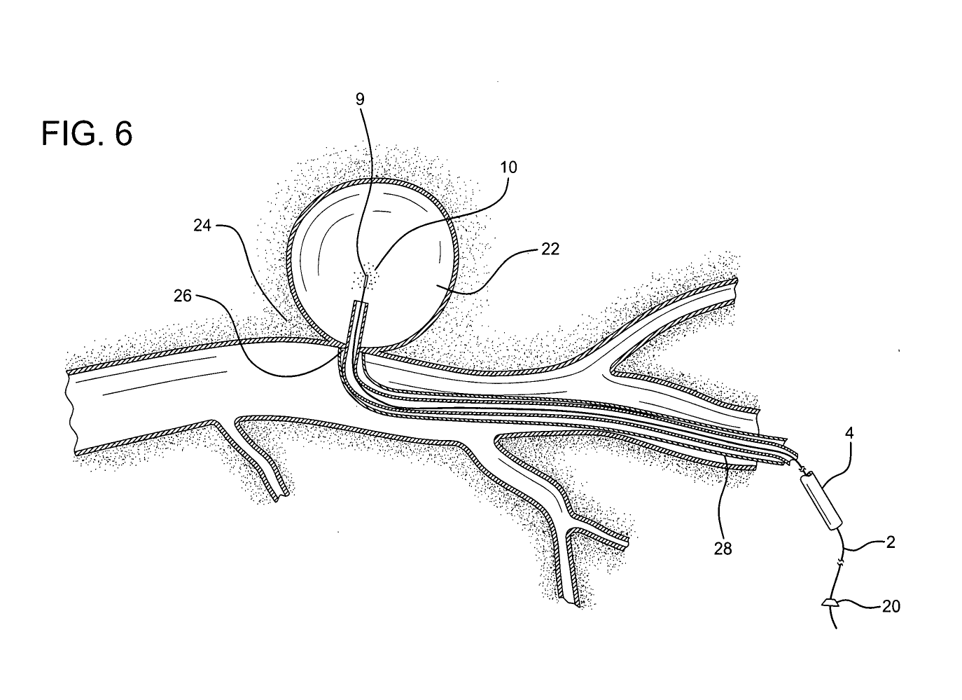

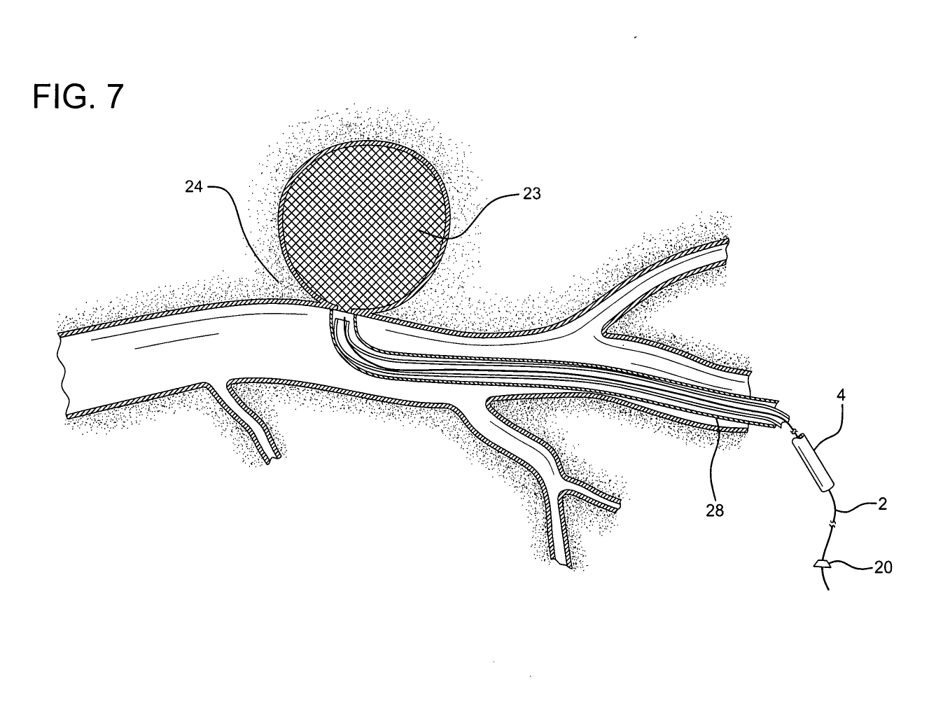

Device 1 of the invention is utilized as follows. After the exact location of the damage or defective vessel, e.g. AVM or aneurysm 22, is determined, catheter 4 containing coated wire 2 is inserted into body 24 of the patient, through preplaced catheter 28, and directed to the damaged tissue 22 and any puncture or lumen 26. (See FIG. 5). Backstop 20 is removed, distal end 8 of catheter 4 is opened, and wire 2, coated with clotting material 10, is pushed or otherwise compelled through and partially out of the catheter to repair or treat the damaged vessel 22. (See FIGS. 3 and 6). The damaged vessel, e.g. the AVM/aneurysm 23 in FIG. 7, is clotted. Catheter 4 and wire 2 are then removed together from the site. (See FIG. 7). The procedure is completed. Optionally, only wire 2 may be removed from the patient and another wire inserted into catheters 4 and 28 to repeat the procedure or for additional activities. The same catheters can be reused.

Alternatively, for superficial pseudoaneurysms such as occurs in the leg where currently, thrombin is directly injected through a hollow gauge needle, a smaller inserter (e.g. a trocar) with its tip coated with clotting material can be inserted into the pseudoaneurysm. Treatment of the pseudoaneurysm avoids the risk of embolization and limb jeopardy which exists with injected thrombin.

Thus the current invention provides a unique system for applying sealants, e.g. thrombin, glue, or other biological clotting sealants like, but not limited to, N butyl cyanoacrylate and EVAC, to treat aneurysms, pseudoaneurysms, and similarly punctured or damaged vessels, while avoiding contact of the sealant with ambient tissue and blood. Targeted clotting occurs in a controlled manner. The system has application in the use of vascular seals in a variety of fields, including but not limited to cerebral, uterine, and coronary.

Certain novel features and components of this invention are disclosed in detail in order to make the invention clear in at least one form thereof. However, it is to be clearly understood that the invention as disclosed is not necessarily limited to the exact form and details as disclosed, since it is apparent that various modifications and changes may be made without departing from the spirit of the invention.

1. A device for sealing wounds in damaged or defective vessels such as aneurysms, pseudoaneurysms, punctured vascular tissue, ateriovenous malformations, fistulas, and other lumens within the body of a patient, the system comprising:
elongated surgical appliance means for delivering clotting material to a damaged and defective vessel, the appliance means having an end tip for the placement of clotting material;
elongated, hollow, micro-sheath means for housing the appliance means;
valve means within the sheath means for allowing the appliance means to pass through the sheath means, the appliance means being positioned within the sheath means such that the sheath means with the valve means at its end tip, provides a protective barrier between the appliance means and non-damaged or defective vessels, preventing clotting material on the end tip of the appliance means from contacting said non-damaged vessels.

2. The device as in claim 1 wherein the appliance means is an elongated thin surgical wire.

3. The device as in claim 1 wherein the sheath means is an elongated catheter.

4. The device as in claim 1 wherein the valve means is a one way valve positioned at the end of the sheath means.

5. The device as in claim 1 wherein the valve means is a petal closure device comprising rotatable plates controlled by wires extending the length of the sheath means.

6. The device as in claim 1 further comprising backstop means to prevent the appliance means from exiting the sheath means.

7. The device as in claim 6 in which the backstop means is located at one end of the sheath means and is removeable from the sheath means, whereby upon its removal the appliance means is permitted to exit the sheath means.

8. The device as in claim 1 wherein the end tip has a straight configuration.

9. The device as in claim 1 wherein the end tip has a spiral configuration.
